(19) United States
(12) Patent Application Publication
(10) Pub. No.: US 2023/0052347 A1
(43) Pub. Date: Feb. 16, 2023

(54) Particle Array Conveying Device and Particle Array Conveying Method

(71) Applicant: Nippon Telegraph and Telephone Corporation, Tokyo (JP)

(72) Inventors: Yuki Hashimoto, Tokyo (JP); Takako Ishihara, Tokyo (JP); Kei Kuwabara, Tokyo (JP)

(21) Appl. No.: 17/793,819

(22) PCT Filed: Feb. 7, 2020
(86) PCT No.: PCT/JP2020/004855

§ 371 Date: Jul. 19, 2022

(51) Int. Cl. B01L 3/00; C12Q 1/02

(52) U.S. Cl. CPC B01L 3/502715; B01L 3/502761; C12Q 1/02; B01L 2300/0645; B01L 2400/0424

(57) ABSTRACT

A particle arrangement transportation device includes: a base material in which is formed a flow channel from an inlet port-side opening through which a solution containing particles to be an object of arrangement and transportation is introduced to an outlet port-side opening; an electrode which is formed along the flow channel on a wall surface of the base material being exposed in the flow channel; electrodes which are formed along the flow channel in the base material on both sides of the flow channel; and a power supply which applies an AC voltage between the electrodes.

### CROSS-REFERENCE TO RELATED APPLICATIONS

This application is a national phase entry of PCT Application No. PCT/JP2020/004855, filed on Feb. 7, 2020, which application is hereby incorporated herein by reference.

### TECHNICAL FIELD

The present invention relates to a particle arrangement transportation device and a particle arrangement transportation method for generating and transporting an arrangement of particles such as bacterial cells.

### BACKGROUND

Techniques for quantitating bacterial cells have a wide range of applications not only in a fundamental research area of biotechnology but also in the fields of medicine, food, hygienic management, and the like (refer to NPL 1). For example, in medical practice, a physical condition of a patient is managed by quantitating bacterial cells contained in skin, mucous membrane, or urine of the patient. In the food sector, a bacterial count is monitored in order to obtain a control guideline of food fermentation. In this manner, a portable bacterial counter that supports on-site measurement is required in various fields.

Conventionally, as methods of quantitating bacterial cells, methods which utilize image recognition such as a colony method and fluorochrome staining and flow cytometry techniques which optically analyze individual bacterial cells have been proposed (refer to NPL 2).

However, the colony method and fluorochrome staining are methods that require cultivation of bacteria while flow cytometry techniques are optical methods and therefore require large-sized apparatuses. Therefore, the methods disclosed in NPL 2 all lack portability.

On the other hand, a recently proposed method analyzes a pattern of electrical pulses flowing through gold micropores embedded with peptides to quantitate bacterial cells that pass through the micropores (refer to NPL 3). According to the method disclosed in NPL 3, a portable bacterial counter capable of detecting individual bacterial cells can be realized.

However, since the method disclosed in NPL 3 requires generating an arrangement of bacterial cells and transporting the arrangement to the micropores in order to detect individual bacterial cells, there is a problem in that an apparatus capable of generating and transporting such an arrangement of bacterial cells is yet to be realized.

### Citation List

### Non Patent Literature

[NPL 1] O. Lazcka, et al., “Pathogen detection: A perspective of traditional methods and biosensors”, Biosensors and Bioelectronics, Vol. 22, pp. 1205-1217, 2007

[NPL 2] R. Hazan, et al., “A method for high throughput determination of viable bacteria cell counts in 96-well plates”, BMC Microbiology, Vol. 12, No. 259, 2012

[NPL 3] M. Tsutsui, et al., “Identification of Individual Bacterial Cells through the Intermolecular Interactions with Peptide-Functionalized Solid-State Pores”, Analytical Chemistry, Vol. 90, pp. 1511-1515, 2018.

### SUMMARY

Embodiments of the present invention have been made in order to solve the problem described above and an object thereof is to provide a particle arrangement transportation device and a particle arrangement transportation method capable of generating an arrangement of particles such as bacterial cells and transporting the arrangement to a specific location.

### Means for Solving the Problem

A particle arrangement transportation device according to embodiments of the present invention includes: a base material in which is formed a flow channel from an inlet port-side opening through which a solution containing particles to be an object of arrangement and transportation is introduced to an outlet port-side opening; a first electrode formed along the flow channel on a wall surface of the base material being exposed in the flow channel; second and third electrodes formed along the flow channel in the base material on both sides of the flow channel; and a power supply configured to apply an AC voltage between the first electrode and the second electrode and between the first electrode and the third electrode.

In addition, a particle arrangement transportation method according to embodiments of the present invention includes, with respect to a particle arrangement transportation device including a base material in which is formed a flow channel from an inlet port-side opening to an outlet port-side opening, a first electrode formed along the flow channel on a wall surface of the base material being exposed in the flow channel, and second and third electrodes formed along the flow channel in the base material on both sides of the flow channel: a first step of applying an AC voltage between the first electrode and the second electrode and between the first electrode and the third electrode; and a second step of introducing a solution containing particles to be an object of arrangement and transportation into the flow channel through the inlet port-side opening.

### Effects of Embodiments of the Invention

According to embodiments of the present invention, by providing a base material in which a flow channel is formed, a first electrode formed along the flow channel on a wall surface of the base material being exposed in the flow channel, second and third electrodes formed along the flow channel in the base material on both sides of the flow channel, and a power supply that applies an AC voltage between the electrodes, an arrangement of particles such as bacterial cells can be generated and transported to a specific location.

### BRIEF DESCRIPTION OF THE DRAWINGS

FIG. 1 is a horizontal sectional view of a particle arrangement transportation device according to an embodiment of the present invention.

FIG. 2 is a plan view of the particle arrangement transportation device according to the embodiment of the present invention.

FIG. 3 is a diagram illustrating a particle arrangement transportation method according to the embodiment of the present invention.

FIG. 4 is a flow chart illustrating the particle arrangement transportation method according to the embodiment of the present invention.

FIG. 5 is a plan view showing an example of an arrangement of particles according to the embodiment of the present invention.

FIG. 6 is a plan view showing another example of an arrangement of particles according to the embodiment of the present invention.

FIG. 7 is a diagram illustrating a method of generating a flow of a solution in the particle arrangement transportation device according to the embodiment of the present invention.

### DETAILED DESCRIPTION OF ILLUSTRATIVE EMBODIMENTS

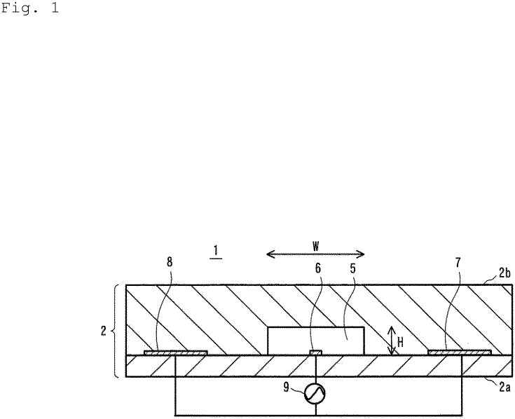

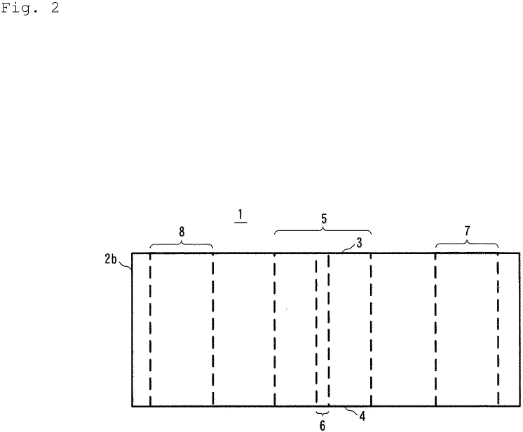

Hereinafter, an embodiment of the present invention will be described with reference to the drawings. FIG. 1 is a horizontal sectional view of a particle arrangement transportation device according to the embodiment of the present invention, and FIG. 2 is a plan view of the particle arrangement transportation device. The present embodiment will be described on the assumption that bacterial cells are spherical particles. A particle arrangement transportation device 1 is constituted of: a base material 2 in which is formed a flow channel 5 from an inlet port-side opening 3 through which a solution containing particles to be an object of arrangement and transportation is introduced to an outlet port-side opening 4; an electrode 6 which is made of a band-like conductor and which is formed along the flow channel 5 on a wall surface of the base material 2 being exposed in the flow channel 5; electrodes 7 and 8 which are made of a band-like conductor and which are formed along the flow channel 5 in the base material 2 on both sides of the flow channel 5; and a power supply 9 which applies an AC voltage between the electrodes 6 and 7 and between the electrodes 6 and 8.

The base material 2 is constituted of a plate-like substrate 2a and a plate-like substrate 2b to be bonded to the substrate 2a. The electrodes 6 to 8 are formed on an upper surface of the substrate 2a. The groove-like flow channel 5 is formed on a lower surface of the substrate 2b at a position where the flow channel 5 covers the electrode 6 when the substrate 2a and the substrate 2b are bonded to each other by removing a lower surface side of the substrate 2b so as to retain an upper surface side thereof. A width W and a height H of the flow channel 5 must be set to sufficiently large values with respect to particles to be an object of arrangement and transportation. A length of the flow channel 5 can be arbitrarily set.

As a material of the substrate 2a and the substrate 2b, materials such as glass, silicon, and plastic can be used.

The substrate 2a and the substrate 2b are bonded so that the lower surface of the substrate 2b comes into contact with the upper surface of the substrate 2a, the flow channel 5 covers the electrode 6, and a lid of the flow channel 5 is closed.

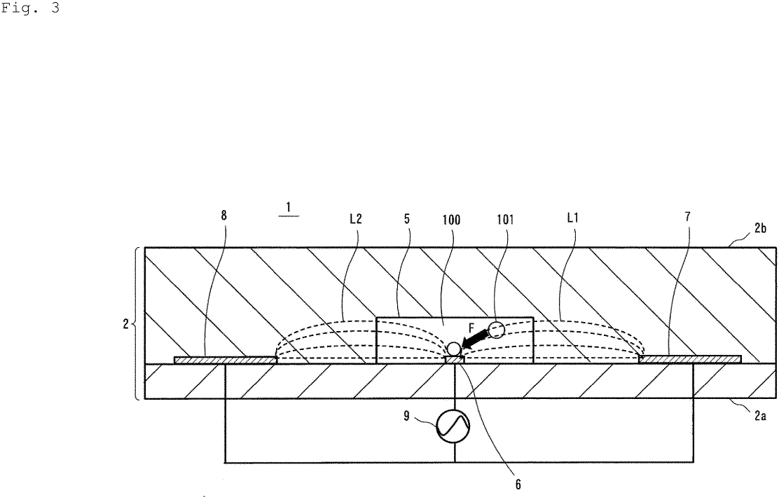

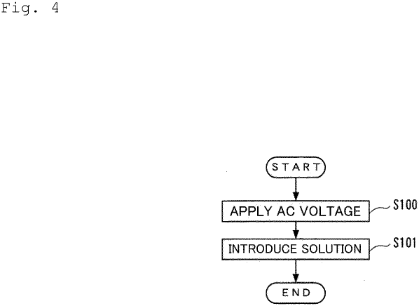

FIG. 3 is a diagram illustrating a particle arrangement transportation method according to the present embodiment, and FIG. 4 is a flow chart illustrating the particle arrangement transportation method.

The power supply 9 applies an AC voltage between the electrode 6 and the electrode 7 and between the electrode 6 and the electrode 8 (step S100 in FIG. 4). In FIG. 3, L1 denotes a line of electric force between the electrode 6 and the electrode 7 and L2 denotes a line of electric force between the electrode 6 and the electrode 8.

In addition, a pump (to be described later) feeds a solution 100 to the inlet port-side opening 3 of the particle arrangement transportation device 1 via a pipe connected to the inlet port-side opening 3 (step S101 in FIG. 4). When the solution 100 containing particles 101 to be an object of arrangement and transportation is introduced to the flow channel 5 from the inlet port-side opening 3, application of the AC voltage described above causes a dielectrophoretic force F shown in expression (1) to act on the particles 101.

$$\text{F} = 2 π {ε}_{s} {r}^{3} R e \left[\frac{{ε}_{p} - {ε}_{s}}{{ε}_{p} + 2 {ε}_{s}}\right] ∇ {\left|E\right|}^{2} \text{.} \text{.} \text{. (1)}$$

In expression (1), εₛ denotes a complex dielectric constant of the solution 100, εₚ denotes a complex dielectric constant of the particles 101, r denotes a radius of the particles 101, E denotes electrical field intensity, and ∇ denotes a nabla operator. Re [] signifies a real part of a complex number described in []. The dielectrophoretic force F is described in reference literature "K. Mogi, et al., “Trapping and isolation of single prokaryotic cells in a micro-chamber array using dielectrophoresis”, RSC Advances, Vol. 6, pp.113000-113006, 2016”.

Since the complex dielectric constants εₛ and εₚ are dependent on a frequency of the AC voltage applied to the electrodes 6 to 8, a direction of the dielectrophoretic force F is also dependent on the frequency of the AC voltage. In the present embodiment, applying an AC voltage with a frequency satisfying εₛ < εₚ to the electrodes 6 to 8 enables the particles 101 to be fixed near the electrode 6 which is a high-electric field region in the flow channel 5.

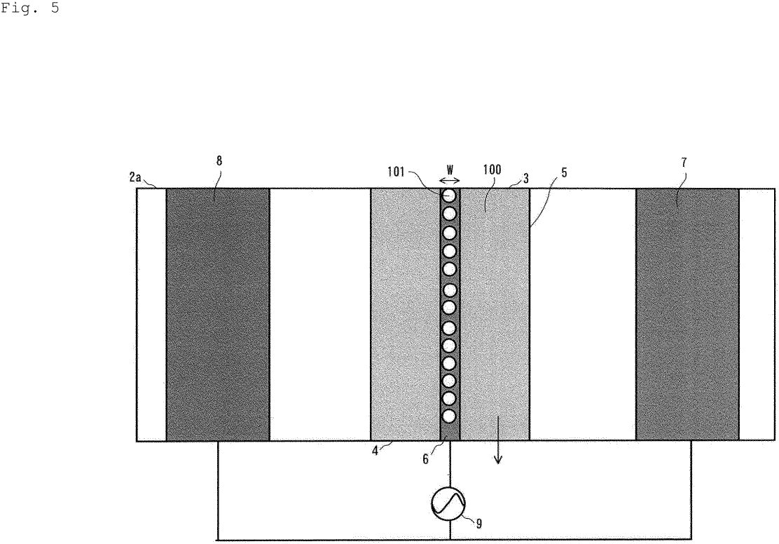

When considering a case where the electrode 6 has a linear shape, since the lines of electric force L1 and L2 when the AC voltage is applied between the electrode 6 and the electrode 7 and between the electrode 6 and the electrode 8 concentrate at edges on both sides of the electrode 6 as shown in FIG. 3, vicinities of the edges on both sides of the electrode 6 become high-electric field regions. Therefore, the particles 101 are more likely to gather near the edges on both sides of the electrode 6. Taking advantage of such characteristics of the particles 101 and, for example, making a width W of the electrode 6 in a direction perpendicular to an extension direction (up-down direction in FIG. 5) of the flow channel 5 and the electrode 6 approximately equal to a diameter of the particles 101 enables an arrangement to be generated in which the particles 101 more or less line up in a single row along the electrode 6 as shown in FIG. 5. Although the particles 101 gather in a vicinity of one of the edges on both sides of the electrode 6, since the width of the electrode 6 is approximately equal to the diameter of the particles 101, an arrangement that is more or less a single row is formed.

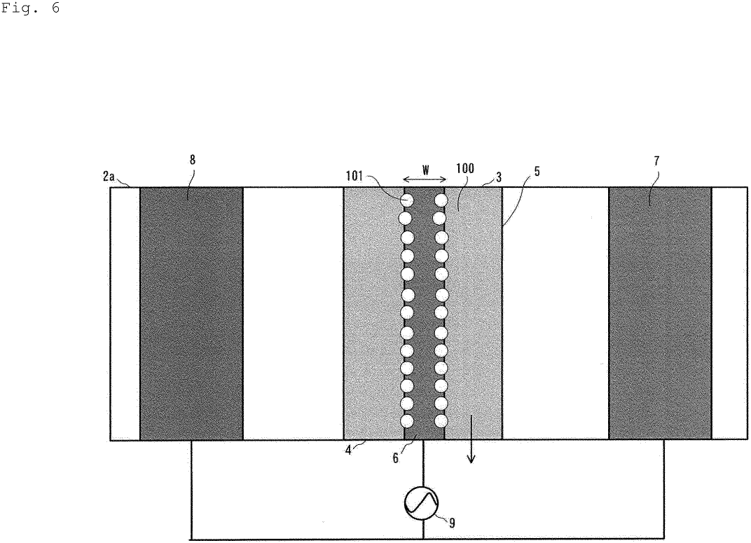

In addition, by increasing the width W of the electrode 6 to twice the diameter of the particles 101 or more, an arrangement in which the particles 101 more or less line up in two rows along the edges on both sides of the electrode 6 can be generated as shown in FIG. 6. Since the particles 101 gather in a vicinity of one of the edges on both sides of the electrode 6 as described above, when the width of the electrode 6 is wide, an arrangement that is more or less two rows is formed.

In this manner, by adjusting a disposition or a size of the electrode 6, an arbitrary arrangement of the particles 101 can be generated.

Generating a flow of the solution 100 enables the arranged particles 101 to be transported in a flow direction of the solution.

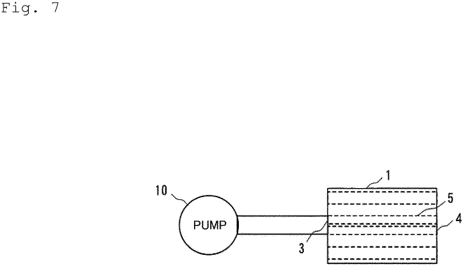

As a method of generating the flow of the solution 100, as shown in FIG. 7, the solution 100 may be fed to the particle arrangement transportation device 1 using a pump 10 that is a peristaltic pump (registered trademark), a syringe pump, or the like.

However, the use of the pump 10 is not an essential constituent element in the present invention. The particle arrangement transportation device 1 may be installed so that the inlet port-side opening 3 is above and the outlet port-side opening 4 is below in order to dispose the flow channel 5 vertically downward or diagonally downward.

As described above, in the present embodiment, an arrangement of particles such as bacterial cells can be generated and transported to a specific location.

Combining the particle arrangement transportation device 1 according to the present embodiment with the sensor disclosed in NPL 3 and causing the particle arrangement transportation device 1 to transport bacterial cells to the micropores of the sensor enables the bacterial cells to be quantitated.

While the present embodiment has been described using bacterial cells as an example of particles, it is needless to say that the present invention can also be applied to particles other than bacterial cells.

### Industrial Applicability

Embodiments of the present invention can be applied to techniques for transporting particles.

### Reference Signs List

1 Particle arrangement transportation device

2 Base material

5 Flow channel

6, 7, 8 Electrode

9 Power supply

10 Pump.

1-7. (canceled)

8. A particle arrangement transportation device, comprising:
a base material comprising a flow channel extending from an inlet port-side opening to an outlet port-side opening, the inlet port-side opening being configured to allow a solution containing particles be introduced;
a first electrode extending along the flow channel on a wall surface of the base material exposed in the flow channel;
second and third electrodes extending along the flow channel in the base material on opposing sides of the flow channel; and
a power supply configured to apply an AC voltage between the first electrode and the second electrode and apply the AC voltage between the first electrode and the third electrode.

9. The particle arrangement transportation device according to claim 8, wherein
the power supply is configured to apply, between the first electrode and the second electrode and between the first electrode and the third electrode, the AC voltage with a frequency that causes a complex dielectric constant of the particles to be larger than a complex dielectric constant of the solution.

10. The particle arrangement transportation device according to claim 8, further comprising:
a pump configured to feed the solution to the inlet port-side opening.

11. The particle arrangement transportation device according to claim 8, wherein the base material is constituted of:
a first substrate with an upper surface on which the first, second, and third electrodes are disposed; and
a second substrate bonded to the first substrate so that a lower surface of the second substrate is in contact with an upper surface of the first substrate, wherein the flow channel is disposed such that the flow channel covers the first electrode when the second substrate is bonded to the first substrate, and wherein the flow channel has a groove shape in a lower surface of the second substrate.

12. The particle arrangement transportation device according to claim 11, wherein the power supply is disposed on an opposing side of the second substrate as the first substrate.

13. A particle arrangement transportation method comprising:
with respect to a particle arrangement transportation device including a base material comprising a flow channel extending from an inlet port-side opening to an outlet port-side opening, a first electrode extending along the flow channel on a wall surface of the base material exposed in the flow channel, and second and third electrodes extending along the flow channel in the base material on opposing sides of the flow channel, a first step of applying an AC voltage between the first electrode and the second electrode and between the first electrode and the third electrode; and
a second step of introducing a solution containing particles to be an object of arrangement and transportation into the flow channel through the inlet port-side opening.

14. The particle arrangement transportation method according to claim 13, wherein
the first step includes the step of applying, between the first electrode and the second electrode and between the first electrode and the third electrode, the AC voltage with a frequency that causes a complex dielectric constant of the particles to be larger than a complex dielectric constant of the solution.

15. The particle arrangement transportation method according to claim 13, wherein
the second step includes the step of feeding the solution to the inlet port-side opening through a pump.

16. The particle arrangement transportation method according to claim 13, wherein the base material is constituted of:
a first substrate with an upper surface on which the first, second, and third electrodes are disposed; and
a second substrate bonded to the first substrate so that a lower surface of the second substrate is in contact with an upper surface of the first substrate, wherein the flow channel is disposed such that the flow channel covers the first electrode when the second substrate is bonded to the first substrate, and wherein the flow channel has a groove shape in a lower surface of the second substrate.

17. The particle arrangement transportation method according to claim 16, wherein the first step includes applying the AC voltage with a power supply that is disposed on an opposing side of the second substrate as the first substrate.
